(19) United States
(12) Patent Application Publication
(10) Pub. No.: US 2016/0310356 A1
(43) Pub. Date: Oct. 27, 2016

(54) DUAL SKIN-CARE SYSTEM

(71) Applicant: Young Han JOO, Ulsan (KR)

(72) Inventor: Young Han JOO, Ulsan (KR)

(21) Appl. No.: 14/692,668

(22) Filed: Apr. 21, 2015

(51) Int. Cl. A61H 23/02; A61F 7/00

(52) U.S. Cl. CPC A61H 23/0263; A61F 7/007; A61F 2007/0087; A61F 2007/0052; A61F 2007/0077; A61H 2205/00; A61H 2201/10

(57) ABSTRACT

A dual skin-care system comprises a skin problem removing device, a skin massaging device, and a power box. The skin problem removing device comprises a main body having a substantially cylindrical shape and an inner through hole, comprising a straight portion, a bent tip portion extending from the straight portion, a pair of lead wires provided through the inner through hole of the main body, and a heating wire attached to the pair of lead wires, disposed so at to be exposed outside through the front opening, and configured for receiving electrical power from the pair of lead wires and generating heat at a tip portion thereof. The skin massaging device comprises a handle, a vibrator, a vibrating portion, and an anion generator provided in the handle and configured for generating and blowing anions into the vibrating portion.

### BACKGROUND OF THE INVENTION

The present invention relates to a dual skin-care system.

Skin is a protecting screen of the body against all kinds of external environment such as five elements and external stimulation or bombardments.

Accordingly, a need for a dual skin-care system has been present for a long time considering the expansive demands in the everyday life. This invention is directed to solve these problems and satisfy the long-felt need.

### SUMMARY OF THE INVENTION

The present invention contrives to solve the disadvantages of the prior art.

An object of the invention is to provide a dual skin-care system.

The dual skin-care system comprises a skin problem removing device, a skin massaging device, and a power box.

The skin problem removing device comprises:

a main body having a substantially cylindrical shape and an inner through hole, wherein the main body comprises a straight portion, a bent tip portion extending from the straight portion, a rear opening provided at a rear end of the straight portion and connected to the inner through hole, and a front opening provided at a front end of the bent tip portion and connected to the inner through hole;

a pair of lead wires provided through the inner through hole of the main body; and

a heating wire attached to the pair of lead wires, disposed so at to be exposed outside through the front opening, and configured for receiving electrical power from the pair of lead wires and generating heat at a tip portion thereof.

The skin massaging device comprises:

a handle;

a vibrator provided in the handle;

a vibrating portion provided at an end of the handle and configured for being driven by the vibrator; and

an anion generator provided on a surface of the vibrating portion and configured for generating and blowing anions into the vibrating portion.

The power box comprises:

a power supply configured for providing electrical power to the pair of lead wires and the heating wire of the skin problem removing device or the vibrator and the anion generator of the skin massaging device;

an adjustable transformer configured for receiving and transforming the electrical power from the power supply and outputting operational power for the skin problem removing device or the skin massaging device; and

a control knob disposed at the rear opening of the main body and configured for controlling the electrical power output from the power supply through and heat at the tip portion of the heating wire of the skin problem removing device by controlling the adjustable transformer or for powering the skin massaging device.

The electrical power through the heating wire is adapted to remove skin problems including age spot, freckle, blemish, and wart.

The skin massaging device is adapted to massage a portion of skin of a user.

The voltage across the tip portion of the heating wire may be configured to be from about 0.7V to about 1.2V and current through the tip portion of the heating wire is configured to be from about 2.5 A to about 3.5 A.

The voltage across the tip portion of the heating wire may be configured to be 0.70V, 0.75V, 0.80V, 0.85V, 0.90V, 0.95V, 1.00V, 1.05V, 1.10V, 1.15V, or 1.20V and the current through the tip portion of the heating wire is configured to be 2.5 A, 2.6 A, 2.7 A, 2.8 A, 2.9 A, 3.0 A, 3.1 A, 3.2 A, 3.3 A, 3.4 A, or 3.5 A.

The voltage across the tip portion of the heating wire may be configured to be 0.91, 0.92, 0.93, 0.94, 095, 0.96, 0.97, 0.98, 0.99, 1.0, 1.2V.

The power supply may comprise a switching mode power supply (SMPS).

The power supply may have an input of AC 100˜260V, 50/60 Hz and an output of AC 0.9˜1.2V, 3 A.

The power supply may have an input of AC 100˜260V, 50/60 Hz and an output of AC 0.94V with the heating wire disconnected.

The power supply may have an output of AC 0.73V and 1.86 A with the heating wire connected.

The power supply may have an input of AC 100˜260V, 50/60 Hz and the voltage across the tip portion of the heating wire is configured to be AC 0.9˜1.2V and about 3.0 A with the heating wire connected.

The power supply may be connected to the pair of lead wires through the adjustable transformer.

The control knob may be electrically connected to the adjustable transformer.

The skin massaging device may be configured to make the vibrating portion vibrate with a controllable frequency.

The bent tip portion may be bent from the straight portion by an obtuse angle.

The obtuse angle may be about 120 degrees.

The power box may further comprise a power output terminal, which is configured for being connected detachably to a power input terminal of the skin problem removing device or the skin massaging device.

The anion generator of the skin massaging device may comprise a plurality of beads made of anion-generating material.

The anion-generating material may comprise elvan.

The beads of the anion-generating material may have a shape of hemisphere or dome.

The plurality of beads may be attached to the surface of the vibrating portion with a specific pattern.

The plurality of beads may be attached in a helical pattern around the surface of the vibrating portion.

The advantages of the present invention are: (1) the dual skin-care system provides a convenient skin problem removing device; and (2) the dual skin-care system provides a skin massaging device, which can be easily switchable.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a diagram showing a dual skin-care system according to an embodiment of the invention;

FIG. 2 is a diagram showing a skin problem removing device and a skin massaging device of a dual skin-care system according to an embodiment of the invention;

FIG. 3 is a diagram showing an inside of a skin problem removing device according to an embodiment of the invention;

FIG. 4 is a diagram showing an inside of a skin massaging device according to an embodiment of the invention;

FIG. 5 is a diagram showing an inside of a skin massaging device according to another embodiment of the invention;

FIG. 6 is a diagram showing an inside of a skin massaging device according to still another embodiment of the invention;

FIG. 7 is a diagram showing a power box according to an embodiment of the invention;

FIG. 8 is a diagram showing a power box according to another embodiment of the invention; and

FIG. 9 is a diagram showing a bottom of the power box of FIG. 8 according to another embodiment of the invention.

### DETAILED DESCRIPTION EMBODIMENTS OF THE INVENTION

Referring to the figures, the embodiments of the invention are described in detail.

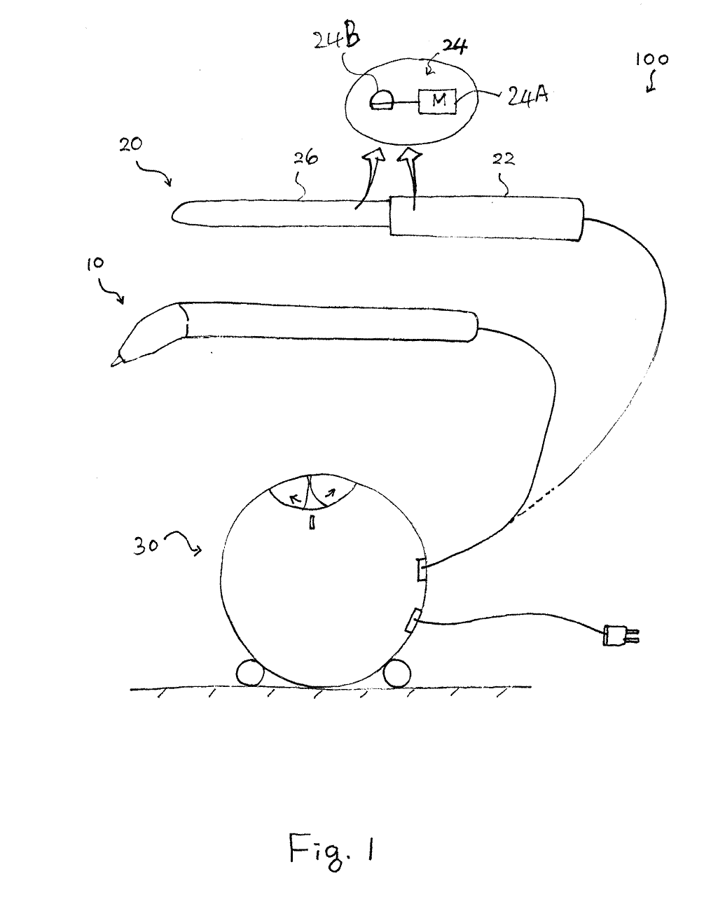

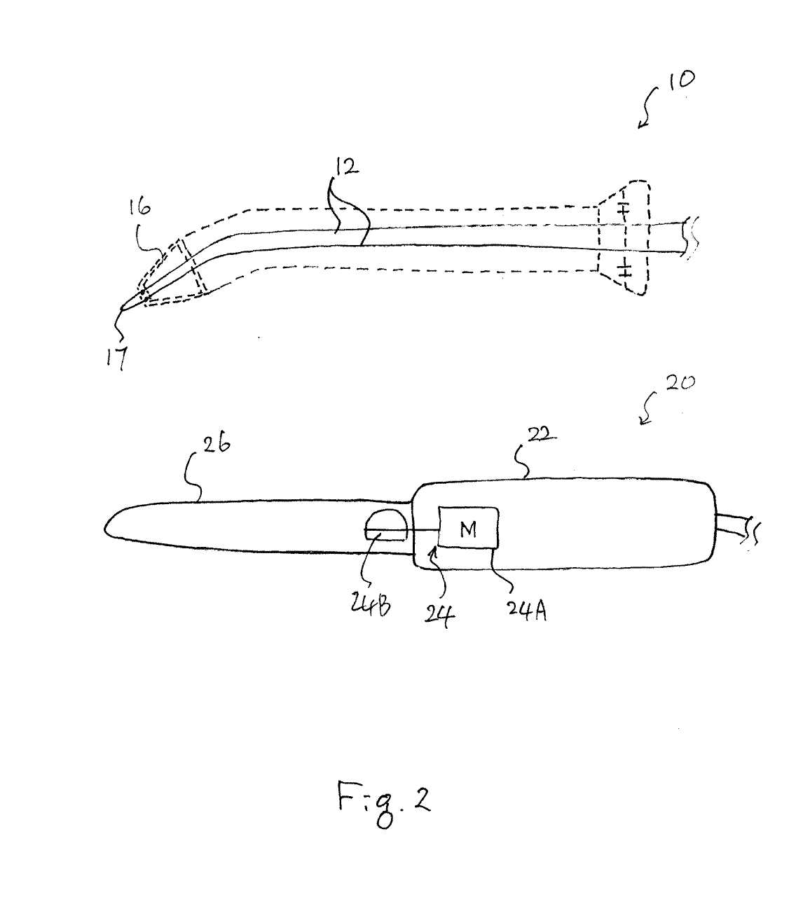

An object of the invention is to provide a dual skin-care system 100 as shown in FIGS. 1 and 2.

The dual skin-care system 100 comprises a skin problem removing device 10, a skin massaging device 20, and a power box 30 as shown in FIG. 1.

The skin problem removing device 10 comprises:

a main body 12 having a substantially cylindrical shape and an inner through hole, wherein the main body comprises a straight portion, a bent tip portion extending from the straight portion, a rear opening provided at a rear end of the straight portion and connected to the inner through hole, and a front opening provided at a front end of the bent tip portion and connected to the inner through hole;

a pair of lead wires 14 provided through the inner through hole of the main body; and

a heating wire 16 attached to the pair of lead wires, disposed so at to be exposed outside through the front opening, and configured for receiving electrical power from the pair of lead wires and generating heat at a tip portion 17 thereof.

The skin massaging device 20 comprises:

a handle 22;

a vibrator 24 provided in the handle;

a vibrating portion 26 provided at an end of the handle and configured for being driven by the vibrator; and

an anion generator 28 provided on a surface of the vibrating portion and configured for generating and blowing anions into the vibrating portion.

The power box 30 comprises:

a power supply 32 configured for providing electrical power to the pair of lead wires and the heating wire of the skin problem removing device or the vibrator and the anion generator of the skin massaging device;

an adjustable transformer 34 configured for receiving and transforming the electrical power from the power supply and outputting operational power for the skin problem removing device or the skin massaging device; and

a control knob 36 disposed at the rear opening of the main body and configured for controlling the electrical power output from the power supply through and heat at the tip portion of the heating wire of the skin problem removing device by controlling the adjustable transformer or for powering the skin massaging device.

The electrical power through the heating wire 16 is adapted to remove skin problems including age spot, freckle, blemish, and wart.

The skin massaging device 20 is adapted to massage a portion of skin of a user.

The voltage across the tip portion 17 of the heating wire 16 may be configured to be from about 0.7V to about 1.2V and current through the tip portion 17 of the heating wire 16 is configured to be from about 2.5 A to about 3.5 A.

The voltage across the tip portion 17 of the heating wire 16 may be configured to be 0.70V, 0.75V, 0.80V, 0.85V, 0.90V, 0.95V, 1.00V, 1.05V, 1.10V, 1.15V, or 1.20V and the current through the tip portion 17 of the heating wire 16 is configured to be 2.5 A, 2.6 A, 2.7 A, 2.8 A, 2.9 A, 3.0 A, 3.1 A, 3.2 A, 3.3 A, 3.4 A, or 3.5 A.

The voltage across the tip portion 17 of the heating wire 16 may be configured to be 0.91, 0.92, 0.93, 0.94, 095, 0.96, 0.97, 0.98, 0.99, 1.0, 1.2V.

The power supply 32 may comprise a switching mode power supply (SMPS).

The power supply 32 may have an input of AC 100˜260V, 50/60 Hz and an output of AC 0.9˜1.2V, 3 A.

The power supply 32 may have an input of AC 100˜260V, 50/60 Hz and an output of AC 0.94V with the heating wire 16 disconnected.

The power supply 32 may have an output of AC 0.73V and 1.86 A with the heating wire 16 connected.

The power supply 32 may have an input of AC 100˜260V, 50/60 Hz and the voltage across the tip portion 17 of the heating wire 16 is configured to be AC 0.9˜1.2V and about 3.0 A with the heating wire 16 connected.

The power supply 32 may be connected to the pair of lead wires 12 through the adjustable transformer 34.

The control knob 36 may be electrically connected to the adjustable transformer 34.

The skin massaging device 20 may be configured to make the vibrating portion 26 vibrate with a controllable frequency.

The bent tip portion may be bent from the straight portion by an obtuse angle.

The obtuse angle may be about 120 degrees.

The power box 30 may further comprise a power output terminal, which is configured for being connected detachably to a power input terminal of the skin problem removing device 10 or the skin massaging device 20.

The anion generator 28 of the skin massaging device 20 may comprise a plurality of beads 29 made of anion-generating material.

The anion-generating material may comprise elvan.

The beads 29 of the anion-generating material may have a shape of hemisphere or dome.

The plurality of beads 29 may be attached to the surface of the vibrating portion with a specific pattern.

The plurality of beads 29 may be attached in a helical pattern around the surface of the vibrating portion.

Referring to FIGS. 1 to 3, the dual skin-care system 100 comprises three parts. The output from the power box 30 may be connected to either the skin problem removing device 10 or the skin massaging device 20 as illustrated. In certain embodiments of the invention, of course, the output from the power box 30 may be connected both to the skin problem removing device 10 and the skin massaging device 20 at the same time. The vibrating portion 26 of the skin massaging device 20 may be operated by the vibrator 24 comprising an electric motor and a cam device configured for generating vibration on the vibrating portion 26 as illustrated. The electric motor 24A and the cam device 24B may be installed in the handle 22 or in the vibrating portion 26 as shown in FIG. 1. Alternatively, the electric motor 24A and the cam device 24B may be installed in the handle 22 and in the vibrating portion 26, respectively, as shown in FIG. 2.

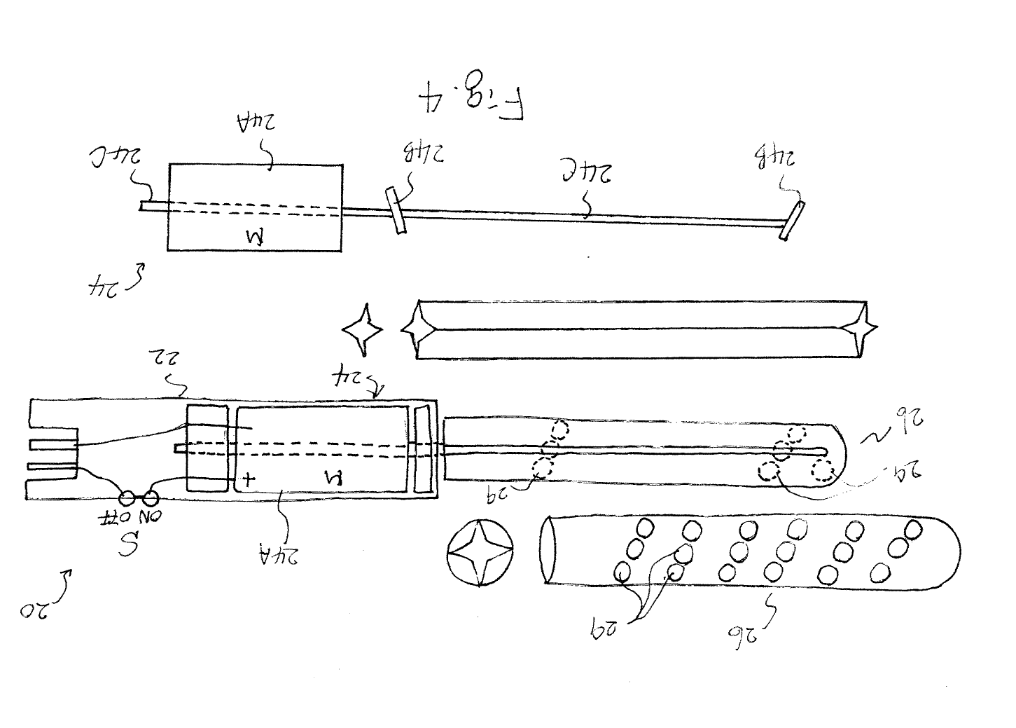

Referring to FIG. 4, the skin massaging device 20 may be provided with a different kind of vibrator 24 as illustrated, in which the plurality of beads 29 provided on a pole through the vibrating portion 26 generates vibration directly, instead of the a cam device that is shown in FIG. 1. The plurality of beads 29 can be provided on a cylindrical body 26 with a four-sided star-shaped through hole, through which a four-sided star-shaped axle may be provided, which engages the rotational axle 24C of the electric motor 24A as illustrated. And, the four-sided star-shaped axle (shown at the second to the last) may have a hole of the same shape therethrough, and the four-sided star-shaped axle and the rotational axle 24C of the electric motor 24A engage each other through two latches 24B. The two latches 24B are provided so as to form a right angle with respect to each other and fit into the four-sided star-shaped axle, locking themselves. And, in this illustrated embodiment, the motor 24A does not provide rotation to the axle, but rocking motion of clockwise and counterclockwise.

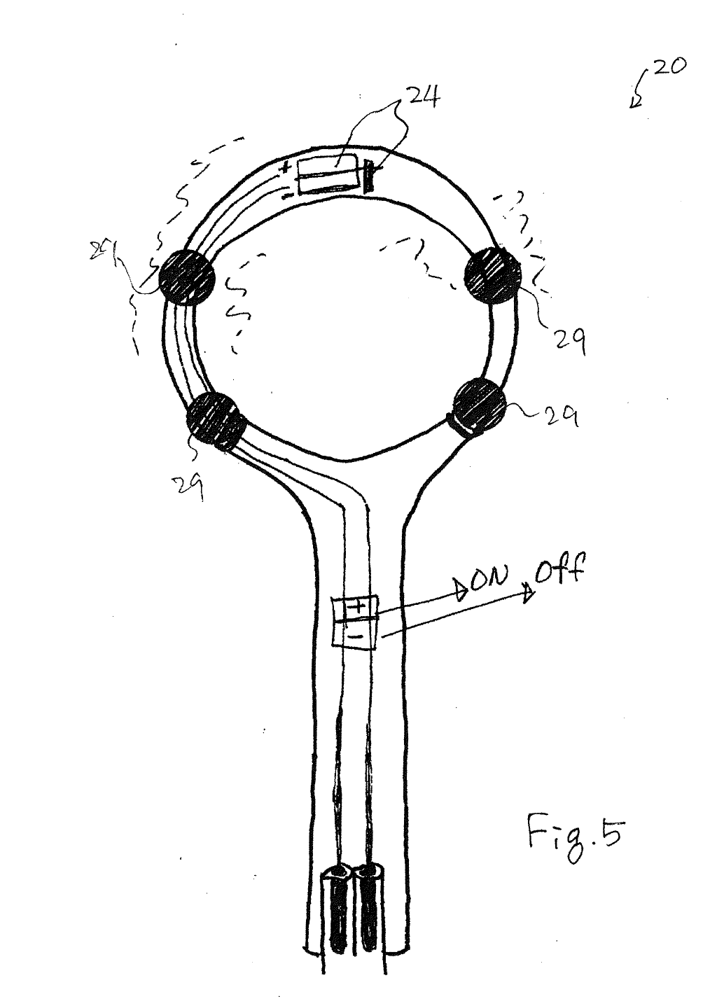

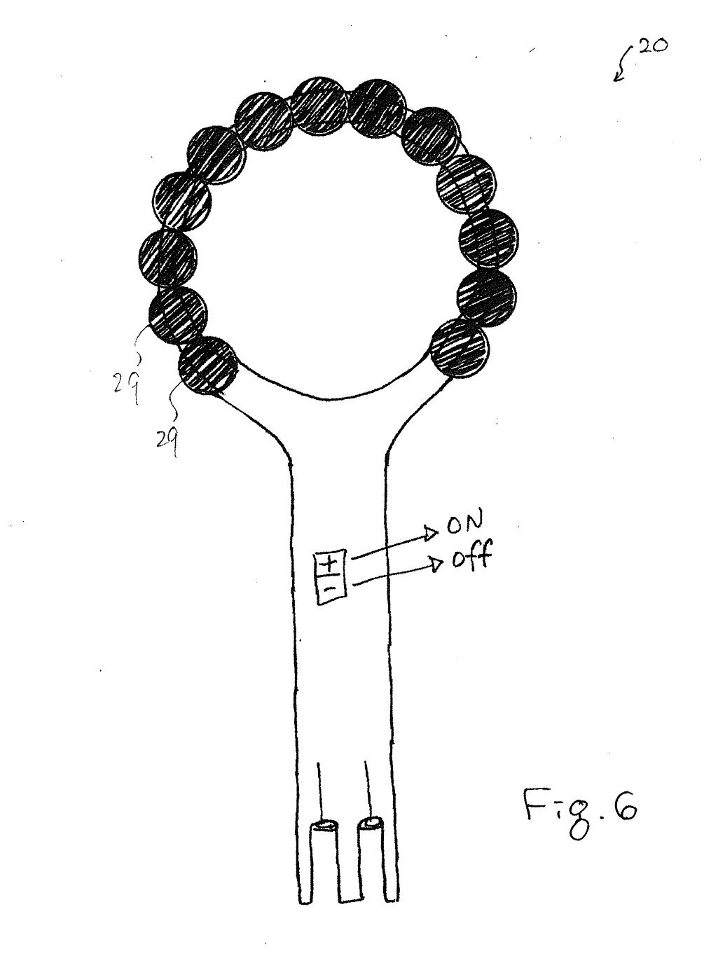

In certain embodiments of the invention as shown in FIGS. 5 and 6, the skin massaging device 20 may be provided in a shape of big ring, and the plurality of beads 29 can be provided over the surface of the ring-shaped body in a specific pattern. In certain embodiments, each of the plurality of beads 29 can have a shape of small ring and be queued through by the big ring as illustrated.

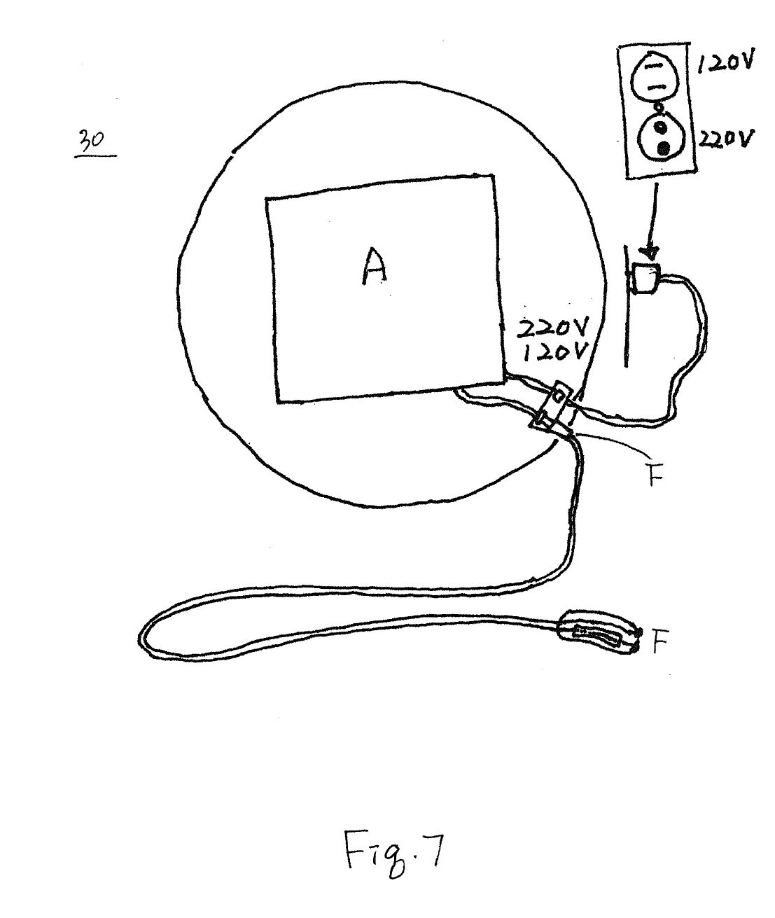

Referring to FIG. 7, the power box 30 may be provided in many designs well known to the community.

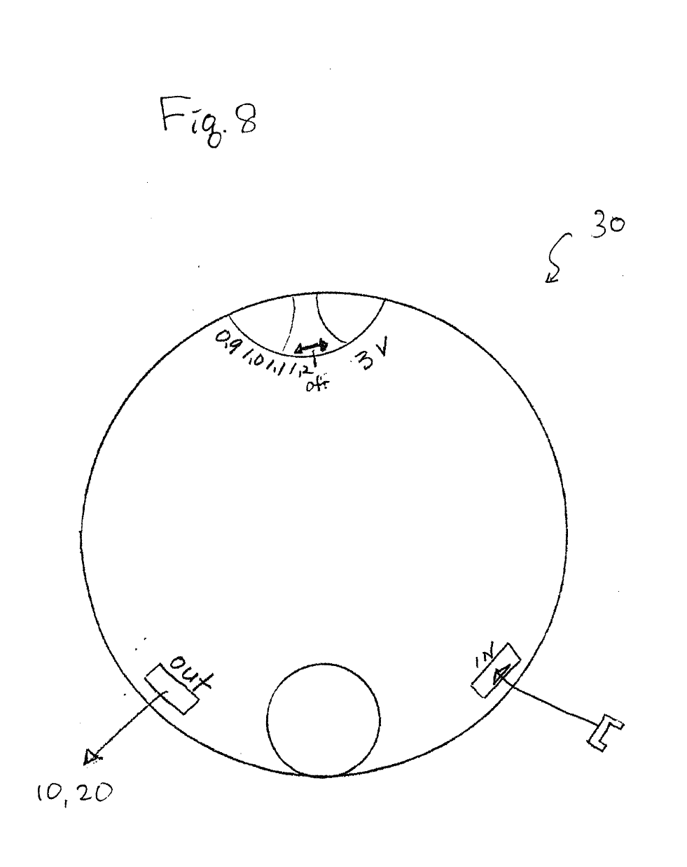

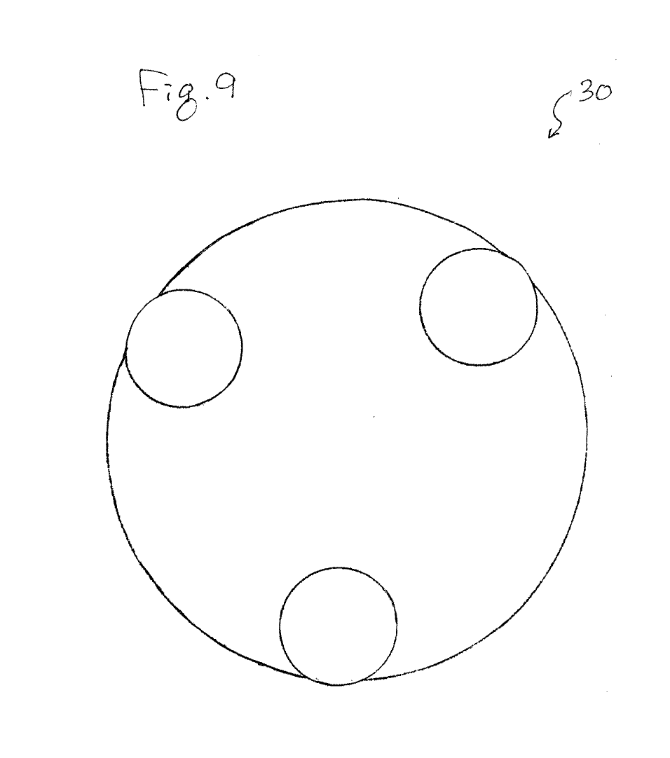

As shown in FIGS. 8 and 9, the power box 30 may be provided in a shape of hemisphere with control knobs on the top surface and three legs on the bottom surface. Alternatively, the power box 30 may be provided in a shape of flattened hemisphere.

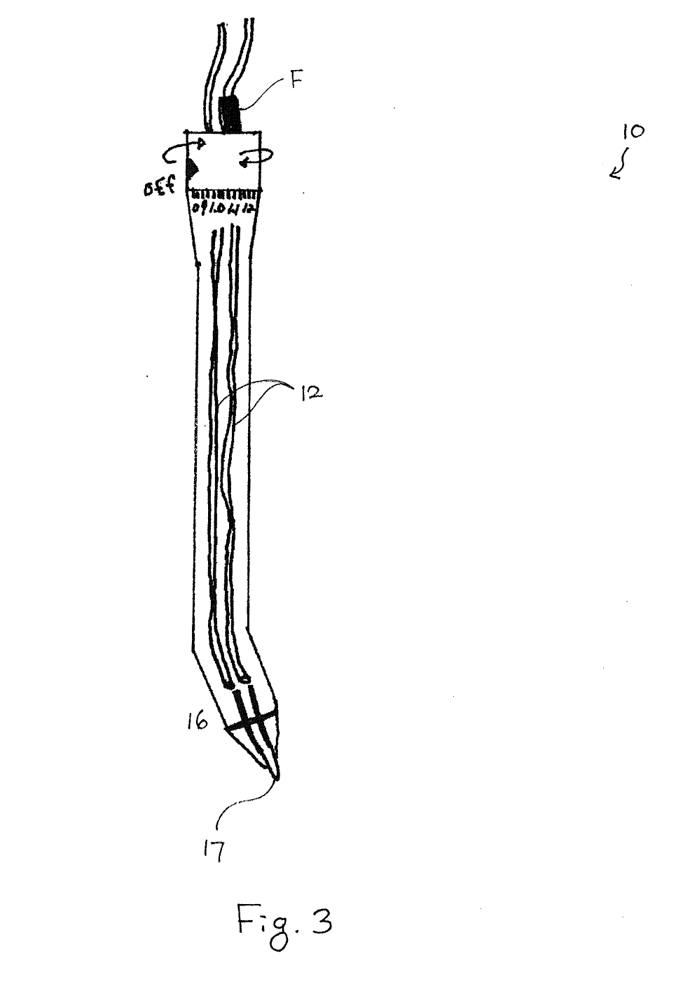

In certain embodiments as shown in FIGS. 2 and 3, the skin problem removing device 10 may have a dial at an end portion thereof for controlling the power, voltage, or current through the tip portion 17 of the heating wire 16 for optimal operation.

As shown in FIG. 7, the power box 30 provides an output with a couple of fuses to protect the circuits.

Also, the skin problem removing device 10 and the skin massaging device 20 may share the power as shown in FIG. 1. Alternatively, they may be connected to the power output of the power box 30.

The power box 30 may be provided in a shape of ball with tripod, in order to enhance stability.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A dual skin-care system comprising:
a skin problem removing device comprising:
a main body having a substantially cylindrical shape and an inner through hole, wherein the main body comprises a straight portion, a bent tip portion extending from the straight portion, a rear opening provided at a rear end of the straight portion and connected to the inner through hole, and a front opening provided at a front end of the bent tip portion and connected to the inner through hole;
a pair of lead wires provided through the inner through hole of the main body; and
a heating wire attached to the pair of lead wires, disposed so at to be exposed outside through the front opening, and configured for receiving electrical power from the pair of lead wires and generating heat at a tip portion thereof;
a skin massaging device comprising:
a handle;
a vibrator provided in the handle;
a vibrating portion provided at an end of the handle and configured for being driven by the vibrator; and
an anion generator provided on a surface of the vibrating portion and configured for generating anion; and
a power box comprising:
a power supply configured for providing electrical power to the pair of lead wires and the heating wire of the skin problem removing device or the vibrator and the anion generator of the skin massaging device;
an adjustable transformer configured for receiving and transforming the electrical power from the power supply and outputting operational power for the skin problem removing device or the skin massaging device; and
a control knob disposed at the rear opening of the main body and configured for controlling the electrical power output from the power supply through and heat at the tip portion of the heating wire of the skin problem removing device by controlling the adjustable transformer or for powering the skin massaging device,
wherein the electrical power through the heating wire is adapted to remove skin problems including age spot, freckle, blemish, and wart, and
wherein the skin massaging device is adapted to massage a portion of skin of a user.

2. The dual skin-care system of claim 1, wherein voltage across the tip portion of the heating wire is configured to be from about 0.7V to about 1.2V and current through the tip portion of the heating wire is configured to be from about 2.5 A to about 3.5 A.

3. The dual skin-care system of claim 2, wherein the voltage across the tip portion of the heating wire is configured to be 0.70V, 0.75V, 0.80V, 0.85V, 0.90V, 0.95V, 1.00V, 1.05V, 1.10V, 1.15V, or 1.20V and the current through the tip portion of the heating wire is configured to be 2.5 A, 2.6 A, 2.7 A, 2.8 A, 2.9 A, 3.0 A, 3.1 A, 3.2 A, 3.3 A, 3.4 A, or 3.5 A.

4. The dual skin-care system of claim 2, wherein the voltage across the tip portion of the heating wire is configured to be 0.91, 0.92, 0.93, 0.94, 095, 0.96, 0.97, 0.98, 0.99, 1.0, 1.2V.

5. The dual skin-care system of claim 2, wherein the power supply comprises a switching mode power supply (SMPS).

6. The dual skin-care system of claim 5, wherein the power supply has an input of AC 100˜260V, 50/60 Hz and an output of AC 0.9˜1.2V, 3 A.

7. The dual skin-care system of claim 5, wherein the power supply has an input of AC 100˜260V, 50/60 Hz and an output of AC 0.94V with the heating wire disconnected.

8. The dual skin-care system of claim 7, wherein the power supply has an output of AC 0.73V and 1.86 A with the heating wire connected.

9. The dual skin-care system of claim 5, wherein the power supply has an input of AC 100˜260V, 50/60 Hz and the voltage across the tip portion of the heating wire is configured to be AC 0.9˜1.2V and about 3.0 A with the heating wire connected.

10. The dual skin-care system of claim 1, wherein the power supply is connected to the pair of lead wires through the adjustable transformer.

11. The dual skin-care system of claim 1, wherein the control knob is electrically connected to the adjustable transformer.

12. The dual skin-care system of claim 1, wherein the skin massaging device is configured to make the vibrating portion vibrate with a controllable frequency.

13. The dual skin-care system of claim 1, wherein the bent tip portion is bent from the straight portion by an obtuse angle.

14. The dual skin-care system of claim 13, wherein the obtuse angle is about 120 degrees.

15. The dual skin-care system of claim 1, wherein the power box further comprises a power output terminal, which is configured for being connected detachably to a power input terminal of the skin problem removing device or the skin massaging device.

16. The dual skin-care system of claim 1, wherein the anion generator of the skin massaging device comprises a plurality of beads made of anion-generating material.

17. The dual skin-care system of claim 16, wherein the anion-generating material comprises elvan.

18. The dual skin-care system of claim 16, wherein the beads of the anion-generating material has a shape of hemisphere or dome.

19. The dual skin-care system of claim 16, wherein the plurality of beads are attached to the surface of the vibrating portion with a specific pattern.

20. The dual skin-care system of claim 19, wherein the plurality of beads are attached in a helical pattern around the surface of the vibrating portion.
